(19) United States
(12) Patent Application Publication
(10) Pub. No.: US 2003/0130700 A1
(43) Pub. Date: Jul. 10, 2003

(54) Optical pulse generator for battery powered photonic pacemakers and other light driven medical stimulation equipment

(76) Inventors: Victor Miller, Clarence, NY (US); Wilson Greatbatch, Akron, NY (US)

Correspondence Address: GREENWALD & BASCH, LLP, 349 WEST COMMERCIAL STREET, SUITE 2490, EAST ROCHESTER, NY, 14445, US

(21) Appl. No.: 10/037,513

(22) Filed: Jan. 4, 2002

(51) Int. Cl. A61N 1/36

(52) U.S. Cl. 607/9

(57) ABSTRACT

An optical pulse generator for battery powered photonic pacemakers and other light driven medical stimulation equipment includes a constant current regulated laser light generator providing a constant controlled light pulse output and constant current control for protection of the laser from current surges. Control is achieved in such manner as to draw practically no current from the host system's battery supply between pulses.

### BACKGROUND OF THE INVENTION

1. Field of the Invention

The present invention relates to photonic pacemakers designed for compatibility with MRI diagnostic equipment, and to other light driven medical stimulation equipment, such as defibrillators, neural stimulators, and the like. More particularly, the invention concerns an optical pulse generator having a constant current regulated laser light generator therein.

2. Description of Prior Art

By way of background, MRI compatible pacemakers for both implantable and wearable use have been disclosed in copending application Ser. Nos. 09/864,944 and 09,865,049, both filed on May 24, 2001, and copending application Ser. Nos. 09/885,867 and 09/885,868, both filed on Jun. 20, 2001. In the aforementioned copending patent applications, each of which names either one or both of applicants as co-inventors, and whose contents are fully incorporated herein by this reference, the disclosed pacemakers feature photonic catheters carrying optical signals in lieu of metallic leads carrying electrical signals in order to avoid the dangers associated with MRI-generated electromagnetic fields. Electro-optical and opto-electrical transducers are used to convert between electrical and optical signals. In particular, a laser diode located in a main pacemaker enclosure is used to convert electrical pulse signals generated by a pulse generator into optical pulses. The optical pulses are carried over an optical conductor situated in a photonic catheter to a secondary housing, where they are converted by a photo diode array into electrical pulses for cardiac stimulation.

Despite the advances in pacemaker MRI compatibility offered by the devices of the copending applications, there remains a problem of how to control and protect the laser diode electro-optical transducer and related control circuitry, while prolonging the pacemaker's battery life. Presently, the current delivered by the pulse generator to the laser diode is not constant over time. It can be influenced by current surges from outside sources, and will steadily drop in any event in accordance with the decreasing voltage output of the pacemaker batteries as they discharge over time. Any reduction in supply current to the laser diode will cause a proportionate reduction in the laser diode's light output, which in turn will drop the power output of the pacemaker relative to the tissue being stimulated. Because the conversion efficiencies of the electro-optical and opto-electrical transducers of a photonic pacemaker are low to begin with, any decrease in the laser diode's light output may produce unacceptable pacemaker performance and is thus of critical concern.

A voltage doubler can be used to compensate for the low end-of-life battery condition. However, the shape of the pulse output of the voltage doubler is not square, and the voltage tends to fall off somewhat during the pulse. This voltage droop reduces the pacemaker power output available from each pulse. Additionally, because the laser diode circuitry tends to draw current from the battery supply in between pulses, battery drain is accelerated and the above power problems are exacerbated.

What is needed is an improvement in the control and protection of a laser diode electro-optical transducer for use in battery powered photonic pacemakers and other light driven medical stimulation equipment. In particular, the light output of the laser diode should be maintained at a constant level notwithstanding current surges and battery discharge. The light output must also be immune to voltage doubler induced pulsatile voltage droop, if a voltage doubler is present. To conserve battery energy and prolong battery life, the laser diode should also be driven in such a way that the control circuitry does not draw appreciable current from the battery supply during the time period between pulses.

### SUMMARY OF THE INVENTION

The foregoing problems are solved and an advance in the art is provided by an optical pulse generator for battery powered photonic pacemakers and other light driven medical stimulation equipment. The optical pulse generator includes an electrical pulse generator and a constant current regulated laser light generator that drives a photonic catheter. The laser light generator is adapted to produce an efficient laser pulse output notwithstanding voltage fluctuations at the output of the electrical pulse generator caused by battery discharge, pulse voltage droop, current surges or other factors. The laser light generator is further adapted to avoid drawing current between pulses provided by the electrical pulse generator.

The electrical pulse generator preferably comprises a voltage doubler, and the laser light generator preferably comprises a laser diode. The laser light generator preferably further includes a transistor that regulates current through the laser diode. The transistor is preferably a junction transistor of the NPN variety and the laser diode is preferably connected in series between the output of the electrical pulse generator and the transistor's collector. The base of the transistor is preferably biased at a substantially constant base biasing voltage using a diode arrangement. This in turn results in the emitter of the transistor being biased at a substantially constant emitter biasing voltage. A collector-emitter current control resistor is connected to the emitter to regulate current through the collector emitter circuit of the transistor. This arrangement provides a constant driving current that powers the laser diode.

### BRIEF DESCRIPTION OF THE DRAWINGS

The foregoing and other features and advantages of the invention will be apparent from the following more particular description of preferred embodiments of the invention, as illustrated in the accompanying Drawing in which:

FIG. 1 is a block diagrammatic view of a battery powered photonic pacemaker;

FIG. 2 is a schematic circuit diagram showing a electrical pulse generator that may be used in the photonic pacemaker of FIG. 1;

FIG. 3 is a schematic circuit diagram showing an electrical pulse generator and voltage doubler that may be used in the photonic pacemaker of FIG. 1; and

FIG. 4 is a schematic circuit diagram showing a constant current regulator that may be used as part of a laser light generator in the photonic pacemaker of FIG. 1, and which is constructed in accordance with a preferred embodiment of the present invention.

### DETAILED DESCRIPTION OF PREFERRED EMBODIMENTS

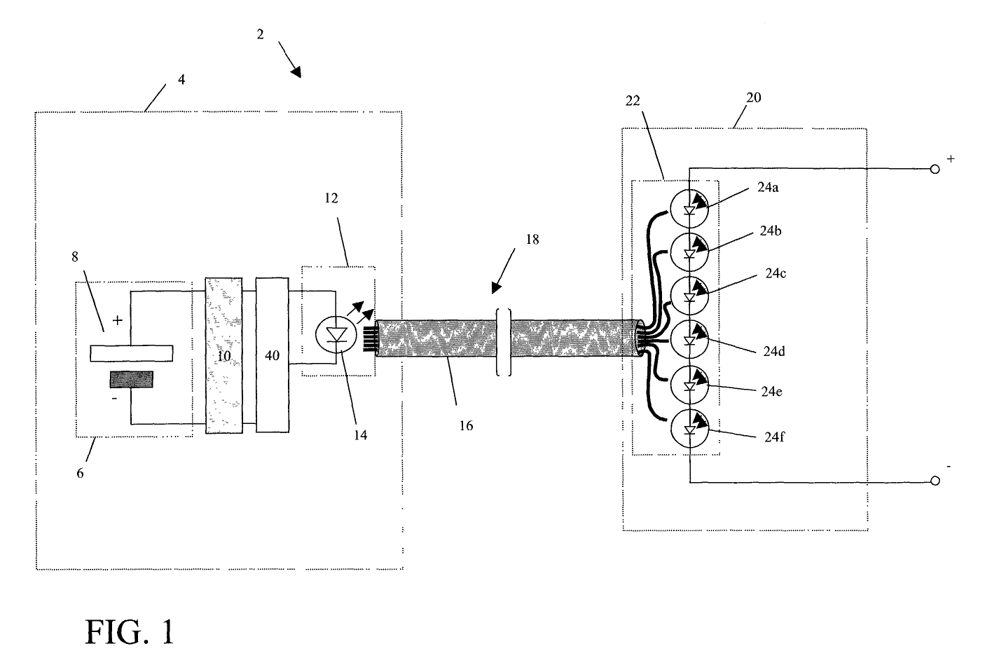

Turning now to FIG. 1, preferred embodiments of the invention will be described within the context of a battery powered photonic pacemaker 2. The pacemaker 2 comprises a main enclosure 4 that may either be implantable or wearable. The main enclosure 4 houses a power supply 6 that comprises one or more batteries 8. In particular, if the main enclosure 4 is designed for implantable use, a single battery 8 designed for implantable service could be used. Examples include conventional lithium iodine batteries (approximately 2.5-4.5 volts) and carbon monofloride batteries (approximately 1.5-3.5 volts). If the main enclosure 4 is designed for external or wearable service, two or three conventional series-connected 1.5 volt batteries 8 could be used. In either case, the power supply 6 will typically provide a steady state d.c. output of at least about 3 volts.

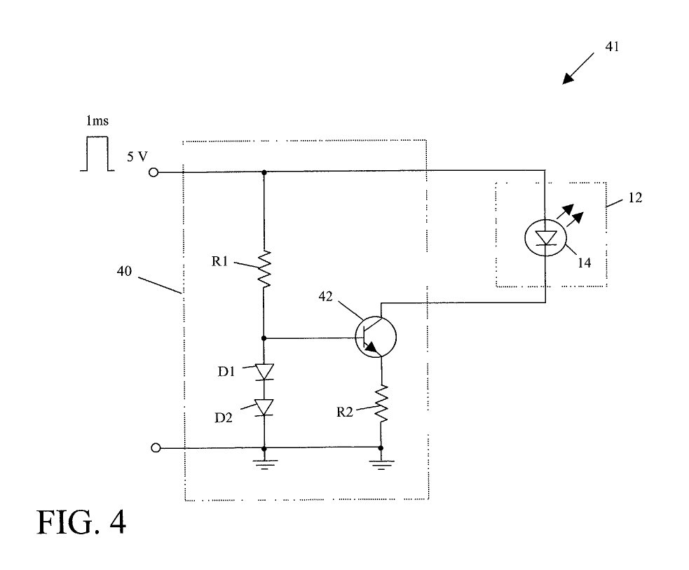

The power supply 6 powers an electrical pulse generator 10 (described in more detail below) that produces electrical pulses at its output. The electrical pulses drive the input of an electro-optical transducer 12, which is preferably implemented using a suitable laser light generator 14, such as a standard 150 milliwatt gallium arsenide laser diode. As will be described in more detail below in connection with FIG. 4, a constant current regulator 40 is disposed between the pulse generator 10 and the electro-optical transducer 12 to control the latter's operation.

The electro-optical transducer 12 generates optical pulses at its output in correspondence with the electrical pulses output by the pulse generator 10. The optical pulses are applied to an optical conductor 16 (preferably a glass fiber optic element) situated in a photonic catheter 18. The photonic catheter 18 extends from the main enclosure 4 to a secondary enclosure 20. There, the optical conductor 16 terminates at an opto-electrical transducer 22 that is preferably implemented as an array of six series-connected photo diodes 24a-24f to develop the required photovoltaic output. The opto-electrical transducer 22 converts the light pulses into electrical pulses which are capable of stimulating the heart.

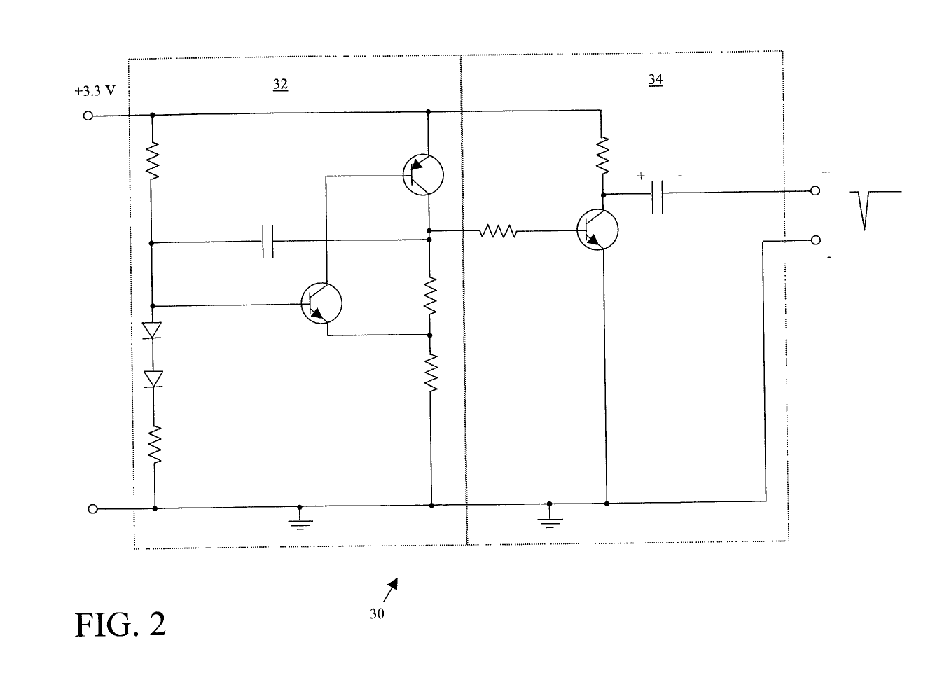

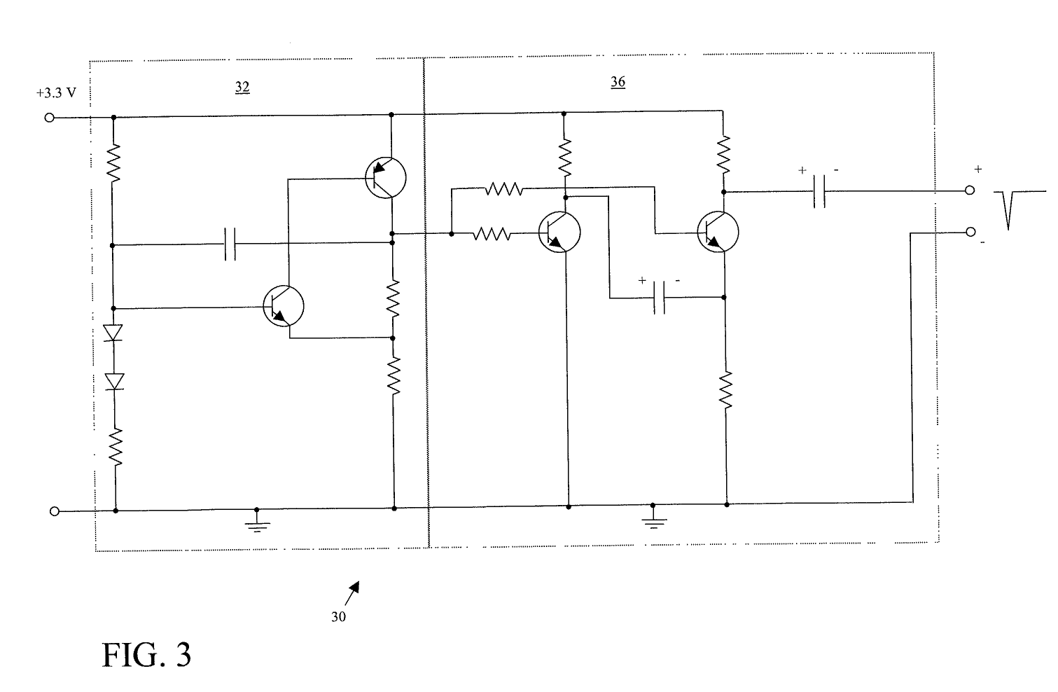

FIGS. 2 and 3 show two alternative circuit configurations that may be used to implement the pulse generator 10. Both alternatives are conventional in nature and do not constitute part of the present invention per se. They are presented herein as examples of the pulsing circuits that have been shown to function well in an implantable pacemaker environment. In FIG. 2, the pulse generator 30 includes an oscillator 32 and an amplifier 34. The oscillator 32 is a semiconductor pulsing circuit of the type disclosed in U.S. Pat. No. 3,508,167 of Russell, Jr. (the '167 patent). As described in the '167 patent, the contents of which are incorporated herein by this reference, the pulsing circuit forming the oscillator 32 provides a pulse width and pulse period that are relatively independent of load and supply voltage. The semiconductor elements are relegated to switching functions so that timing is substantially independent of transistor gain characteristics. In particular, a shunt circuit including a pair of diodes is connected so that timing capacitor charge and discharge currents flow through circuits that do not include the base-emitter junction of a timing transistor. Further circuit details are available in the '167 patent. The values of the components which make up the oscillator 32 can be selected to provide a conventional VOO pacemaker pulses varying from about 0.1-10 milliseconds duration at a period of about 1000 milliseconds.

The amplifier 34 of FIG. 2 is a circuit that uses a single switching transistor and a storage capacitor to deliver a negative-going pulse of approximately 3.3 volts across the pulse generator outputs when triggered by the oscillator 32. An example of such a circuit is disclosed in U.S. Pat. No. 4,050,004 of Greatbatch (the '004 patent), which discloses voltage multipliers having multiple stages constructed using the circuit of amplifier 34. As described in the '004 patent, the contents of which are incorporated herein by this reference, the circuit forming the amplifier 34 uses a 3.3 volt input voltage to charge a capacitor between oscillator pulses. When the oscillator 32 triggers, it drives the amplifier's switching transistor into conduction, which effectively grounds the positive side of the capacitor, causing it to discharge through the pulse generator's outputs. The values of the components which make up the amplifier 34 may be selected to produce an output potential of about 3.3 volts.

The amplifier 36 of FIG. 3 is a circuit that uses a pair of the amplifier circuits of FIG. 2 to provide voltage doubling action. As described in the '004 patent, the capacitors are arranged to charge up in parallel between oscillator pulses. When the oscillator 32 triggers, it drives the amplifier's switching transistors into conduction, causing the capacitors to discharge in series to provide the required voltage doubling action. The values of the components that make up the amplifier 36 may be selected to produce an output potential of about 6.6 volts.

With reference now to FIG. 4, an exemplary embodiment of the constant current regulator 40 is shown. The purpose of the constant current regulator 40 is to controllably drive the electro-optical transducer 12 using the electrical pulse output of the pulse generator 10 (see FIG. 1). Collectively, the constant current regulator 40 and the electro-optical transducer 12 provide a constant current regulated laser light generator 41 in accordance with the invention. The current regulator 40 of FIG. 4 uses an NPN transistor 42 arranged in a common emitter configuration to drive the laser diode 14. A suitable NPN transistor that may be used to implement the transistor 42 is a switching transistor given by the designation 2N4401. As stated above, the laser diode 14 can be implemented as a standard 150 milliwatt gallium arsenide laser diode, and this is assumed to be the case in the circuit diagram of FIG. 4. The recommended power level for driving such a device is about 100 milliwatts. The required input voltage is about 2 volts. Assuming there is a conventional diode voltage drop of about 0.7 volts across the laser diode 14, a driving current of about 140 milliamps should be sufficient to achieve operation at the desired 100 milliwatt level (0.7 volts×140 milliamps=98 milliwatts). However, as stated by way of background above, the current through the laser diode 14 must be relatively constant to maintain the desired power output. The constant current regulator 40 achieves this goal.

In particular, the base side of the transistor 42 is biased through a resister R1 and a pair of diodes D1 and D2. The diodes D1 and D2 are connected between the base of the transistor 42 and ground. Each has a conventional diode voltage drop of about 0.7 volts, such that the total voltage drop across the diodes D1 and D2 is about 1.5 volts and is substantially independent of the current through the diodes (at operational current levels). This means that the base of the transistor 42 will be maintained at a relatively constant level of about 1.5 volts notwithstanding changes in the input voltage supplied from the pulse generator 10. The value of the resistor R1 is selected to be relatively high to reduce the current draw through the base of the transistor 42. By way of example, a value of 2500 ohms may be used for R1. Assuming a supply voltage of about 5 volts, as represented by the input pulse waveform in FIG. 4, the current through the resistor R1 will be a negligible 1.4 milliamps ((5-1.5) volts/2500 ohms).

Importantly, the emitter side of the transistor 42 will remain at a relatively constant level of about 1 volt (assuming a base-emitter voltage drop across the transistor 42 of about 0.5 volts). A resistor R2 is placed between the emitter of the transistor 42 and ground in order to establish a desired current level through the collector-emitter circuit of the transistor 42. Note that this also represents the driving current through the laser diode 14 insofar as the laser diode is connected in series between the current regulator's supply voltage (the output of pulse generator 10) and the collector of the transistor 14. Because the voltage potential at the transistor emitter is about 1 volt, if R2 is selected to be a 7 ohm resistor, the resultant current level will be about 1 volt/7 ohms=140 milliamps. This corresponds to the current level required to drive the laser diode 14 at the desired operational power level.

It will thus be seen that the current through the laser diode 14 is primarily dependent on the value of R2 and is substantially unaffected by changes in supply voltage. The transistor 42 will deliver a constant current pulse of about 140 ma to the laser diode 14 when driven into conduction by a pulse from the pulse generator 10 (see FIG. 1). The pulse length may vary from about 0.1 ms to 10 ms, depending on the pacing requirements of the patient. In the event that the voltage at the pulse generator's output pulse begins to drop at the onset of a an end-of-life battery condition, the pulse current seen by the laser diode 14 will remain relatively constant. In this way the laser diode's light output will be carefully controlled. The laser diode 14 will likewise be protected from effects of voltage surges cause by outside sources.

Note that the supply voltage delivered by the pulse generator 10 (see FIG. 1) must be higher than the 2 volts required to drive the laser diode 14 insofar as the transistor emitter sits at approximately 1 volt. As a result, when an end-of-life battery condition arises, the available voltage may become marginal. Thus, it will generally be preferable to use the voltage doubling amplifier 36 of FIG. 3 to drive the constant current regulator 40. This will allow the pulse generator to deliver a pulse of about 5 volts or more, which should be more than sufficient to drive the laser diode 14. Note, however, that the shape of the pulse provided by the voltage doubling amplifier 36 is not square and the voltage falls somewhat during each pulse. Advantageously, however, the constant current regulator 40 will compensate for this droop and will supply a relatively constant current to the laser diode 14, thereby insuring a constant light output during the pulse.

An additional advantage of the constant current regulator 40 is that the laser diode 14 will not draw current between pulses due to the transistor 42, which will be in a cut-off mode between each pulse.

Accordingly, an optical pulse generator has been disclosed for use with battery powered photonic pacemakers and other light driven medical stimulation equipment. While various embodiments of the invention have been shown and described, it should be apparent that many variations and alternative embodiments could be implemented in accordance with the invention. For example, it will be appreciated that alternative circuit configurations could be used to implement the constant current regulator 40, including circuits with temperature compensating components, etc. In addition, although the constant current regulator 40 is shown in the context of a photonic pacemaker, it could be implemented in any light driven medical stimulation system wherein photonic pulses are delivered for medical use. Such devices include, but are not limited to, defibrillators, neural stimulators, and other medical equipment designed to stimulate body tissue using either electrical current or direct application of light energy. It is understood, therefore, that the invention is not to be in any way limited except in accordance with the spirit of the appended claims and their equivalents.

I claim:

1. An optical pulse generator for battery powered photonic pacemakers and other light driven medical stimulation equipment, comprising:
an electrical pulse generator;
a constant current regulated laser light generator for driving a photonic catheter;
said laser light generator being connected to an output of said electrical pulse generator;
said laser light generator being adapted to produce an efficient optical pulse notwithstanding voltage fluctuations at said electrical pulse generator output caused by battery discharge, pulse voltage droop, current surges or other factors; and
said laser light generator being further adapted to avoid drawing current between pulses provided by said electrical pulse generator.

2. An optical pulse generator in accordance with claim 1, wherein said electrical pulse generator comprises a voltage doubler.

3. An optical pulse generator in accordance with claim 1, wherein said laser light generator comprises a laser diode.

4. An optical pulse generator in accordance with claim 3, wherein said laser light generator comprises a transistor, and wherein said laser diode is driven through a collector of said transistor.

5. An optical pulse generator in accordance with claim 4, wherein a base of said transistor is biased at a substantially constant base bias voltage notwithstanding changes in supply voltage at said electrical pulse generator output.

6. An optical pulse generator in accordance with claim 5, wherein said transistor base bias voltage is maintained by a diode arrangement extending between said transistor base and a common voltage reference.

7. An optical pulse generator in accordance with claim 6, wherein a current limiting resistor is provided between said transistor base and said electrical pulse generator output.

8. An optical pulse generator in accordance with claim 7, wherein an emitter of said transistor is maintained at a relatively constant voltage level by virtue of said relatively constant transistor base bias voltage, and wherein a resistor is provided between said emitter and said common voltage reference to establish a substantially constant driving current through said laser diode.

9. An optical pulse generator in accordance with claim 4, wherein said transistor is a bipolar junction transistor.

10. An optical pulse generator in accordance with claim 9, wherein said transistor is an NPN transistor.

11. A battery powered photonic pacemaker system, comprising:
an electrical pulse generator;
a constant current regulated laser light generator for driving a photonic catheter;
said laser light generator being connected to an output of said electrical pulse generator;
said laser light generator being adapted to produce an efficient optical pulse notwithstanding voltage fluctuations at said electrical pulse generator output caused by battery discharge, pulse voltage droop, current surges or other factors; and
said laser light generator being further adapted to avoid drawing current between pulses provided by said electrical pulse generator.

12. A battery powered light driven medical stimulation system, comprising:
an electrical pulse generator;
a constant current regulated laser light generator for driving a photonic catheter;
said laser light generator being connected to an output of said electrical pulse generator;
said laser light generator being adapted to produce an efficient optical pulse notwithstanding voltage fluctuations at said electrical pulse generator output caused by battery discharge, pulse voltage droop, current surges or other factors; and
said laser light generator being further adapted to avoid drawing current between pulses provided by said electrical pulse generator.
